(19) United States
(12) Patent Application Publication
(10) Pub. No.: US 2018/0235722 A1
(43) Pub. Date: Aug. 23, 2018

(54) CORDLESS AND WIRELESS SURGICAL DISPLAY SYSTEM

(71) Applicant: GSI GROUP, INC., San Jose, CA (US)

(72) Inventors: Amir BAGHDADI, Gilroy, CA (US); Chad CHANG, Fremont, CA (US); Lam HUYNH, Pleasanton, CA (US); Daniel ORNELAS, San Jose, CA (US); Jonah POST, Campbell, CA (US); Rainer SCHOLL, Saratoga, CA (US); Darko SPOLJARIC, Seattle, WA (US)

(21) Appl. No.: 15/117,975

(22) PCT Filed: Mar. 8, 2016
(86) PCT No.: PCT/US16/21363

§ 371 Date: Aug. 10, 2016

Related U.S. Application Data
(60) Provisional application No. 62/253,606, filed on Nov. 10, 2015.

(51) Int. Cl. A61B 90/00; A61B 17/00

(52) U.S. Cl. CPC A61B 90/37; A61B 2017/00734; A61B 2090/372; A61B 2017/00221

(57) ABSTRACT

A cordless wireless and mobile display system having an integrated system for delivering uninterrupted power to a display is described. The cordless wireless and mobile display includes wireless capabilities that allows it to receive and transmit information from associated medical devices and to send data to other designated devices. A battery assembly includes a primary and secondary battery that supplies uninterrupted power to a display. Sensors periodically sample the power output from the battery in current use to ensure that enough power is available to the display. When sensors detect power output levels have fallen below a preset value, indicators alert users that the system is switching to a backup power supply and that the primary battery supply needs recharging.

### CROSS REFERENCE TO RELATED APPLICATIONS

This application claims priority to U.S. Provisional Patent Application No. 62/253,606 filed Nov. 10, 2015 titled “CORDLESS AND WIRELESS SURGICAL DISPLAY SYSTEM”.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The present invention relates generally to a wireless and cordless mobile medical display system, and more particularly, to a wireless and cordless mobile medical display system able to receive and/or transmit information having battery backup system for powering the wireless and cordless surgical mobile display systems.

### BACKGROUND

Monitoring devices and displays make modern day surgeries more predictable and routine by providing visual assistance to the surgical team. Most standard surgical displays require an electrical cord for powering the display and associated components. Having to move the surgical display along with its corresponding components can become cumbersome during surgery especially if the surgical display is plugged into a wall socket for power and there is need to maneuver the surgical display around other surgical monitoring and support devices that are also physically tethered to a power supply in the wall or on a power strip. It would also be advantageous to have a display system that did not receive its power through a cord tethered to the wall and that was easily maneuverable around an operating room as well as from room to room.

Equally important, it would be advantageous to have a surgical display unit that has wireless capabilities where it is able to wirelessly receive and/or transmit information to and from other medical components or support devices. While wireless capabilities have been expanded to a plethora of devices, many systems used in a medical setting are still dependent upon the standard signal wire to connect medical devices to each other. It would be especially useful to be able to transmit information in the way of images or biological parameters, as an example, during a medical procedure so that it can be easily shared with members of the medical team performing the procedure or even to an outside party that may aid in diagnosing or advising on a procedure. Not only would having less wires crisscrossing a medical procedure room advantageous in itself from a safety and cleanliness standpoint, now a patient may also have the aid of medical experts elsewhere advising on their procedure.

While having a surgical display that does not have a physical power cord is advantageous as mentioned above, there are considerations that must be addressed for a cordless and wireless surgical display that would not be an issue for a unit having a power cord. With a cordless surgical display, the main concern is the battery life of the unit especially when the surgery is longer than anticipated. Losing the ability to view the area on which the surgical procedure is being performed on may cause set-backs and delays in the procedure as well as increased risk of complication and infection in the patient. Thus, there is a need for a cordless surgical display that contains a secondary power source.

Next, having to swap out the battery in the cordless surgical monitor during surgery also introduces risks to the patient. For one, the battery may become drained and need to be replaced during a critical time during the surgery when the loss of visual of the area being operated on can cause major impediments. In some instances, there is very minimal warning or no warning at all that the battery is about to become drained. Often times, there is a break or interruption in the power during the switching of the batteries such that the display is no longer showing the images that the surgical team may need to proceed with the procedure. Also when the surgical display fails due to the battery drain, a member of the surgical team might be pulled away from his surgical duties to restore the power for the surgical display which places a strain on the team in general. Alternatively, a separate hospital staff not involved in the surgery may be asked to restore power to the surgical display, but not without increasing the risk of contaminating the sterile environment. Thus, there is a need for a cordless surgical display that is able to sense when the first battery is about to become drained and indicate that the system is switching to a secondary battery.

In addition, it would be advantageous to have a completely non-tethered display for surgical or medical purposes. This would not only include cordless unit but also a display having wireless capabilities. Having both features would allow freedom of movement of these displays within the procedure room or between hospital rooms.

In light of the complications associated with having a corded surgical display and with current cordless surgical displays, there is a need for improving the battery system that will have the capability to automatically switch over to a secondary power source without losing the ability to transmit and display the image.

### SUMMARY OF THE DISCLOSURE

The present invention relates to a wireless and cordless mobile display system having a smart battery system. It is the object of this present invention to have a mobile display that is able to wirelessly receive information, such as video images, that then may be displayed on a display panel of the wireless and cordless mobile display system.

It is another object of this present invention to provide a wireless and cordless mobile display system that is able to both transmit and receive information, such as video images. In the receive mode, the wireless and cordless mobile display system may receive video as well as data information wirelessly, then display this information on the display panel while in a transmit mode, the wireless and cordless mobile display system may send data, such as images, to completely external components, such as a second display panel.

It is an object of this present invention to provide a cordless mobile display system for use in a surgical procedure room having a display panel, components for detecting, receiving, and transmitting signals to and from the cordless and wireless display system, a battery assembly that provides power to the wireless display panel, a battery controller, and a battery charging station. The cordless and wireless mobile display system also includes a mobile stand for supporting the wireless display panel, the receiving and transmitting components, the battery assembly, and the battery charging station.

It is yet another object of the present invention to provide a smart battery system for powering surgical displays. The smart battery system that know when the available remaining power of the primary battery falls below a pre-set level and to switch from the primary battery to a secondary battery as the power source without a break in supplying power to the surgical display. The smart battery system may be capable of determining and displaying the percent capacity of the primary battery and the secondary battery including the remaining run time of each battery respectively.

The battery assembly in the previously-described cordless mobile display system provides power to the cordless display and includes a primary battery and a secondary battery. The primary battery provides power to the cordless display under normal operating conditions. The secondary battery is able to power the cordless mobile display system when the power output from the primary battery falls below a predetermined level.

The battery controller is able to monitor the power output from the primary battery and the secondary battery. The battery controller is able to switch the power from the primary battery to the secondary battery when the primary battery power output falls below a pre-determined power output level. When required, the battery controller is able to switch power from the primary battery to the secondary battery in 10 milliseconds or less and without interrupting power to the surgical display system. The battery controller is also able to charge the primary and the secondary battery when required. Further, the battery controller is able to measure and display the time associated with the remaining power stored within the primary and the secondary battery.

It is another object of this present invention to provide a cordless and wireless mobile display system that will alert the user, either audibly or visually, that one of the following is about to occur or occurring: the primary battery has reached a pre-determined power output level and the system is about to switch to the secondary battery, the primary battery is charging, when the primary battery power has dropped to a predetermined level that may be unable to support all the display and periphery components and the system is switching the power output from the primary battery to the secondary battery, the battery controller is switching power from the secondary battery back to the primary battery, the secondary battery is recharging, or both the primary and the secondary battery have reached the pre-determined power level and the system will be without power in approximately “x” amount of time.

It is yet another object of this present invention to provide a cordless and wireless mobile display system that will have the capability to transmit and receive data from other surgical components or units being used in the particular procedure or from an outside source, such as the hospital's database having information pertinent to the particular patient. The transmission may be through physical wires that link the cordless mobile display system to the other surgical components or the connection may be through wireless means.

The wireless and cordless mobile display system may also retain information on the primary and the secondary batteries. This may include the length of time that it takes each of these batteries to decline to the predetermined level where then the system switches over to the alternative battery or the length of time required to charge the batteries. This information is useful for determining when the batteries may need to be replaced. The cordless mobile display system may also have a USB connection for transmitting information the status of the batteries or other information related to the cordless wireless display

It is another object of this present invention that the cordless mobile display system be easily maneuverable. The cordless mobile display system can have a wheeled base of at least two or more wheels.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is a pictorial of a first embodiment of a cordless and wireless mobile display system.

FIG. 2 is a close-up of a wireless display panel having a receiver and transmitter.

FIG. 3 is a pictorial of wireless transmission components that may be used with the cordless and wireless display system.

FIG. 4 is a block diagram depicting a video display assembly of the cordless and wireless mobile display system.

FIG. 5 is a block diagram depicting a battery controller and associated components of the cordless and wireless mobile display system.

FIG. 6 is a pictorial of a second embodiment of a cordless and wireless mobile display system.

FIG. 7 is a close-up pictorial of a battery assembly having a primary and a secondary battery for the second embodiment of the cordless and wireless mobile display system

FIG. 8 is a pictorial of a recharging unit for the first and the second battery.

### DETAILED DESCRIPTION

Described herein are system and devices for displaying aspects of a medical procedure, such as during surgery, where the device is cordless and utilizes wireless connections for receiving and transmitting information. The information may be in the way of images or potentially any data related to the procedure being performed. In general, the cordless and wireless mobile display system includes a display panel, a video controller, a user interface, a battery assembly and a battery controller. In some embodiments, the cordless and wireless display mobile system may also include USB adapters and ports for storing, receiving, and displaying images and other relevant data to and/or from other the components.

Firstly, the cordless and wireless display mobile systems described herein are cordless display systems. The term cordless refers to electrical devices that are powered by a battery or battery pack. These devices can be operated without a power cord that would connect the device to an electrical supply such as an outlet, generator, or other power source.

Next, the display system described herein is also wireless. The term wireless should not be confused with and used interchangeably with the term cordless here. The term wireless, generally refers to a device having the ability to transfer information to and from other devices between two or more points where the points are not connected by an electrical connector. The information may be in a video format or be any form of data. The cordless and wireless mobile display system in one aspect is able to wirelessly receive information such as video images, from a medical component, such that the information (video images in this case) may be displayed on the display panel. The information that is received or transmitted will be in real time, such that a medical professional will be able to see what he or she is doing at the procedure site instantaneously on the display. In another aspect, the cordless and wireless mobile system is able to wirelessly transmit information, such as images, to a secondary device, such as a secondary display, to be viewed by other members of the medical procedure team. The term “mobile” while often used interchangeable with the term “wireless” in everyday use, is used to here to mean highly moveable because it is un-tethered and may be repositioned with ease.

The cordless and wireless display system includes a display panel. Suitable display panels may be LCD (liquid crystal display panel) type display panels. Other possible display panels include plasma display panels, OLEDs (organic light emitting display panels), surface conduction electron-emitter display (SED), field emission display (FED), laser TV, displays incorporating carbon nanotubes, quantum dot display panels, and interferometric modulator display panels. The system may also be interfaced with a three dimensional display panels such as a sweep-volume display panel, a varifocal mirror display panel, an emissive volume display panel, laser display panels, holographic display panels, and light field display panels. While the majority of medical monitors are simplistic and only display a fairly straightforward signal, such as a heartbeat or some other biological parameter, the cordless and wireless display system's display panel component conceived of herein may be a high resolution display that is able to discern and display fine details of a medical procedure.

The cordless and wireless display system also includes rechargeable primary and secondary batteries. Rechargeable batteries are those that can be charged, discharged into a load, and recharged many times over. Rechargeable batteries are composed of one or more electrochemical cells. Different combination of electrode materials and electrolytes may be used. Suitable compounds include but are not limited to: lead-acid, alkaline, nickel-iron, nickel hydrogen, nickel zinc, nickel cadmium (NiCd), nickel metal hydride (NiMH), lithium cobalt oxide, lithium ion, lithium iron phosphate, lithium sulfur, lithium titanate, lithium ion polymer, sodium ion, thin film lithium, zinc bromide, zinc cerium, vanadium redox, sodium sulfur, molten salts, silver oxide, and quantum batteries.

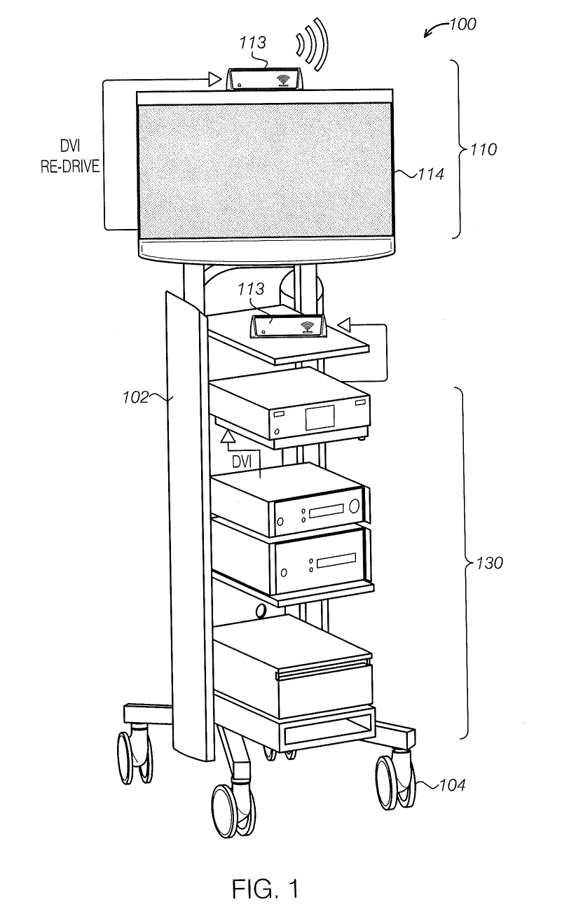

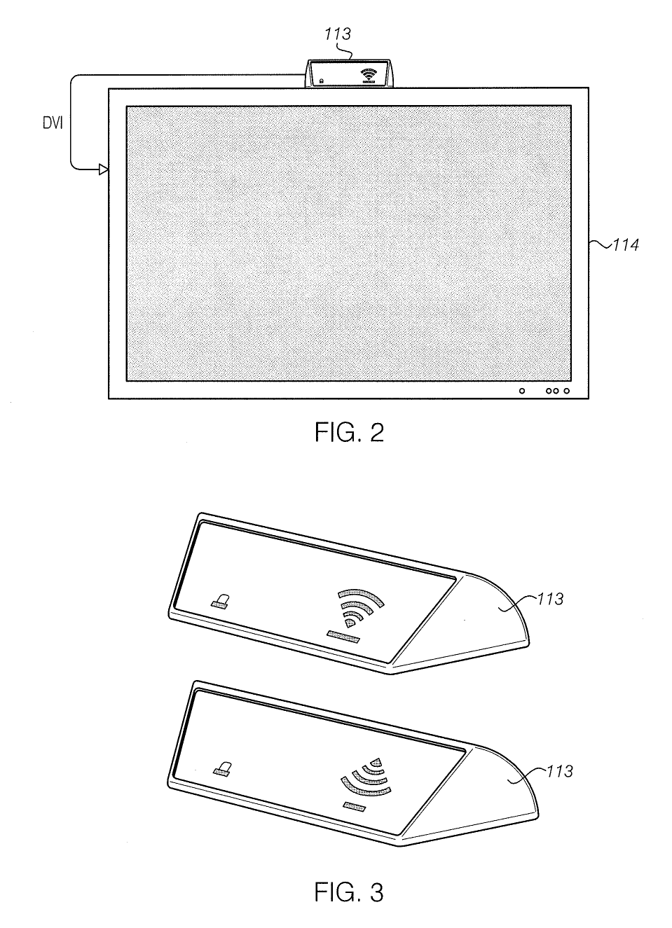

Turning to FIGS. 1 and 2, a first embodiment of a cordless and wireless mobile display system 100 will be described. Cordless and wireless mobile display system 100 includes a mobile stand 102 that supports wireless display assembly 110, a battery assembly 120, and associated video components 130. While cordless and wireless mobile display system 100 may have wires that connect wireless display assembly 110 to the other components within the cordless and wireless mobile display system 100, the system is designated as wireless because cordless and wireless mobile display system 100 is able to receive and/or transmit information via wireless means through a wireless transmitter/receivers 113 (e.g., the ZeroWire® wireless video solution available from NDS Surgical Imaging, Inc.) and examples are discussed below. Thus, cordless and wireless display 100 has the advantage over display units currently available that require a physical power cord to power the system as well as having physical connections between it and other medical monitoring units that are needed during any particular surgical procedure. The plethora of cables and wires emanating from the medical display restricts the ability to maneuver the display during a procedure and also increase the possibility that wires may be miss-connected or accidentally uncoupled when shifted.

As previously alluded to, the cordless and wireless mobile display system 100 may include medical components connected to or integrated within its system. As an example, FIG. 1 actually includes camera components for an endoscope. In this example, the endoscope may be able to send images of the areas that it is viewing within the patient back to cordless and wireless mobile display system 100 where the images may be displayed on the display panel 114. Thus, it is conceivable to also include other medical components with cordless and wireless mobile display system 100 that are used to image, detect, or measure some treatment area on a patient during a procedure where the image or other data information is then transmitted back to the cordless and wireless mobile display system 100 and then ultimately shown on display panel 114.

The cordless and wireless mobile display system 100 also includes transmitting capabilities where it is able to transmit received data, such as image data, to a separate, external component. The external component may be a second display panel, data processing or retention device. For example, it may be useful during a surgical procedure for multiple members of the surgical team to have access to what is being sensed and wirelessly transmitted to the cordless and wireless mobile display system. It is not feasible for the members of the surgical team to all crowd around a single display and the proximity of a single display may not allow for easy viewing by other medical team members that may benefit from viewing the images or data. The cordless and wireless mobile display system 100 will be able to transmit data, such as image data, to periphery display panels or other devices such that all surgical team members that would benefit from viewing the images or data will have access to that information.

Staying with FIG. 1, the battery assembly 120 is not shown, but it should be noted that the battery assembly 120 is located on the body of the cordless and wireless system 100. In this example, the battery assembly 120 powers the components of the cordless and wireless mobile display system 100 through physical wires or connections. Mobile stand 102 includes a wheel assembly 104 for maneuvering display system 100 to the desired location. Wireless display assembly 110 may be wireless with the aid of commercially available wireless accessories that are associated with it, but in this present embodiment, wireless display assembly 110 may be wired to associated video components 130 in a conventional way.

Associated video components 130 may be any components utilized during a medical procedure, including, but not limited to monitors, sensing units, measuring devices and so forth. In the actual FIG. 1, the associated video components 130 shown are an imaging device and a lighting component. As mentioned, a variety of different analyses and monitoring components may be included in the associated display components. In this configuration, associated video components 130 are coupled to and held in place on the cordless and wireless mobile display system 100 by any suitable fasteners such as bolts, screws, pins, straps, and so forth. Associated video components 130 may be physically wired to other components within cordless and wireless mobile display system 100 but may also include wireless capabilities for sending and receiving information. In more generalized terms, video components 130 may be any medical component needed to be coupled to the wireless mobile display system 100 for a particular medical procedure.

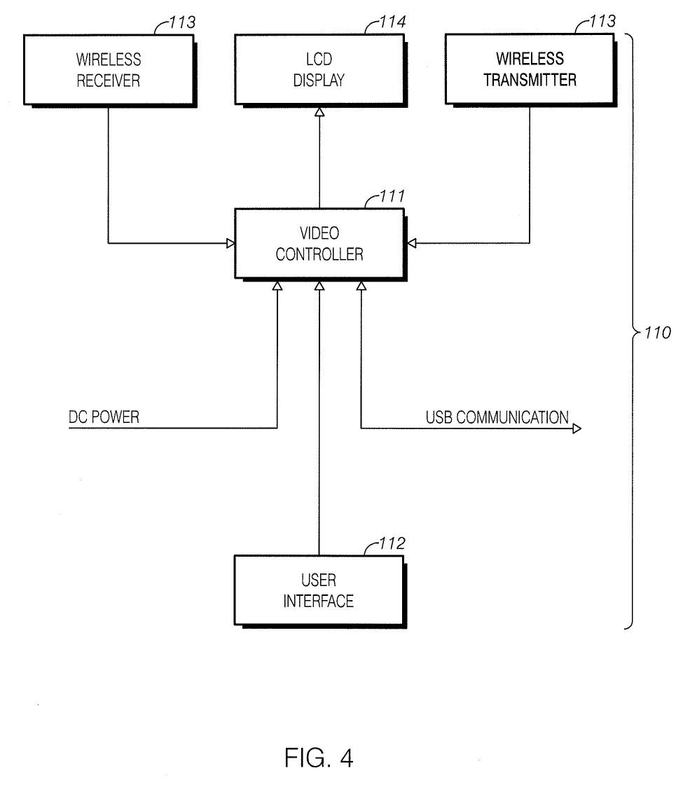

FIG. 4 shows a block diagram of the high level components of the wireless display assembly 110. Wireless display assembly 110 includes a display panel 114, a video controller 111, and a user interface 112. The display panel 114 may be any type of suitable display, such as CRT, LCD, LED, OLED, and so forth. As previously eluded to, video controller 111 is in electrical or signal communication with the wireless receiver and/or transmitter 113, both of which are part of the wireless display assembly 110 and may be housed together or be in separate, but coupled containments. Wireless display assembly 110 may also be in wireless or wired connected to the remaining components of the cordless and wireless mobile display system 100. Finally, any user interfaces may be incorporated into the wireless and cordless display system such as touchpad or touch sensor, buttons, knobs, keys, toggles, and so forth for adjusting the display panel 114.

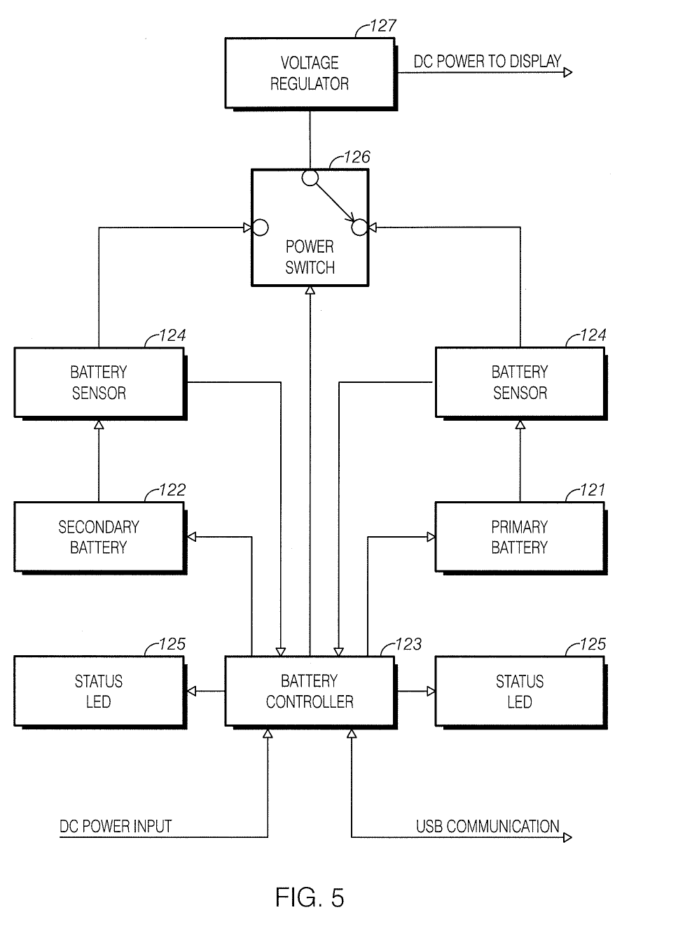

Battery assembly 120 includes both a primary battery 121, a backup or secondary battery 122, and a battery controller 123 all of which can be seen in FIG. 5. Battery assembly 120 also includes sensors 124 for detecting the amount power that either primary battery 121 or secondary battery 122 can output. Also included in battery assembly 120 are status indicators 125 for showing the user the status of primary battery 121 and secondary battery 122. Cordless and wireless mobile display system 100 may also include a voltage regulator 127 to ensure that the necessary amount of power reaches the display at all times during use. In some examples, the status indicators can also display the amount of battery life left for either the primary or secondary battery in terms of hours and minutes or on a meter. In other examples, the secondary battery becomes the primary battery once the original primary battery's reserves are depleted.

In use, cordless and wireless mobile display system 100 start out being powered by primary battery 121. The output provided is delivered through a power switch 126. Power switch 126 is controlled by battery controller 123 and is the physical switch that switches the output that powers the display between primary battery 121 and secondary battery 122. Initially power switch 126 is set to receive power from primary battery 121. As primary battery 121 outputs power to the display, its corresponding battery sensor 124 periodically measures its power output and relays this information to battery controller 123. The time between when battery sensor 124 takes a power output measurement may be preset within the system or may be set by the user. Reasonable sampling times for the battery sensors may be every few minutes, every five minutes, every ten minutes, every fifteen minutes, and so forth.

After receiving the output values from battery sensor 124, battery controller 123 will compare the value sent to a pre-determined value set with the system. When battery controller 123 detects an output power value that is equal to or fall below the pre-determined value, battery controller 123 will signal power switch 126 to divert its power connection away from primary battery 121 and to secondary battery 122, where secondary battery 122 will now supply the power to wireless display assembly 110 and associated video components 130. The time battery controller 123 takes to switch between primary battery 121 and secondary battery 122 and back is unnoticeable by the user, typically on the order of ten milliseconds or less. Because of this short switch over time, there is no detectable interruption in the images displayed. In some examples, cordless and wireless display system may also provide either or both an audible signal and a visual signal that it is switching battery output from its primary battery to its secondary battery, alerting the user that primary battery is in need of recharging.

Cordless and wireless mobile display system 100 displays the status of both primary battery 121 and secondary battery 122. In some examples the cordless and wireless display system may have a separate display panel or screen for displaying battery status. In other examples, the cordless and wireless display system may have a USB port where a user can access the status of either the primary or secondary battery. In some other examples, the status of the primary and the secondary batteries may be accessed wirelessly.

Cordless and wireless mobile display system 100 also includes associated video components 130 that support and provide an interface between wireless display assembly 110 and the previously discussed components of cordless and wireless mobile display system 100. As previously mentioned, wireless display assembly 110 is powered by battery assembly 120 and will receive data, such as video data, from the associated video components 130. Associated video components 130 also draw power from battery assembly 120.

Wireless components 113 allow cordless and wireless mobile display system 100 to transmit and receive video signals and other pertinent data. For example, medical diagnostic tools such as endoscope capsules or cameras can transmit data to the cordless and wireless display system to be displayed so that healthcare professionals may be better able to visualize the area being attended to. Also, the cordless and wireless display system is able to transmit images to other wireless systems including computers, mobile devices, and other medical diagnostic assemblies.

For cordless and wireless mobile display system 100, wireless transmitter and receiver components 113 are external to the corresponding system as shown in FIG. 1. FIG. 3 shows external wireless transmitter and receiver that can be coupled to the cordless and wireless display system for providing wireless capabilities to the system. In other examples, the wireless receiver and transmitter may be partially or wholly contained within the system. This will be more thoroughly discussed below.

Video controller 111 is in electrical communication with user interface 112 as well as battery assembly 120. Video controller 111 draws power from battery assembly 120. In some examples, where there is no separate display for showing the status of battery assembly 120, video controller 111 maybe receive and send battery assembly 120 status to wireless display assembly 114 to be shown. Also, video controller 111 may be able to receive status information about battery assembly 120 through a USB port or other suitable connection (with the battery assembly 120).

User interface 112 allows the user to configure the cordless and wireless display system to be in a receive or a transmit mode. In some examples, the user interface can be the display screen where the display screen is an interactive, touch sensitive screen. In other examples, the user interface may be a separate key pad or series of keys, buttons, knobs, switches, or the like that allow the user to select whether the cordless and wireless display system is in a receive or a transmit mode, adjust the display and control other aspects of the cordless and wireless display system.

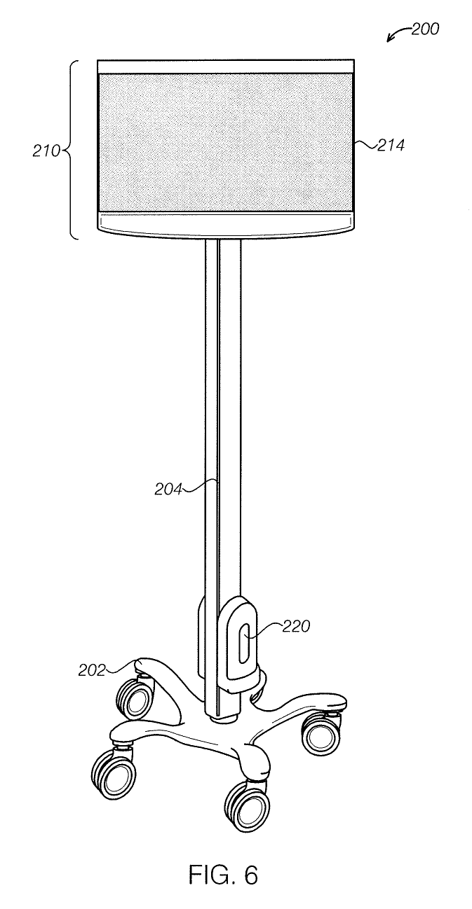

Turning to FIGS. 6-8, an alternative configuration of the cordless and wireless mobile display system 100 is shown and labeled as cordless and wireless display 200. Cordless and wireless mobile display system 200 may share similarities with the previously described cordless and wireless mobile display system 100 embodiment and thus, certain features of cordless and wireless mobile display system 200 will not be redundantly described.

Cordless and wireless mobile display system 200 functions in a similar manner as cordless and wireless mobile display system 100. One major difference between cordless and wireless mobile display system 200 as compared to cordless and wireless mobile display system 100 is that the former configuration has many more relevant medical components (such as diagnostic or detection components) that are integrated internally or wirelessly coupled to the system. In the former scenario, some or all the wireless and/or video components for cordless and wireless mobile display system 200 may be integrated into its body (i.e. into the stem, the display assembly, and/or the stand). In some cases, the associated display components may also be designed to fit within the body of the cordless and wireless mobile display system 200. In other instances, the associated medical components (such as camera, diagnostic, or detection components) may be external to the cordless and wireless mobile display system 200 but still wirelessly-tethered to each other such that the other associated medical components is able to transmit data, such as images, to the cordless and wireless mobile display system 200. Like cordless and wireless mobile display system 100, cordless and wireless mobile display system 200 is also able to transmit information, such as images, to other devices for image data to be displayed on a secondary display panel or to have data information transmitted to an external device.

Cordless and wireless mobile display system 200 is also more compact and streamlined compared to cordless and wireless mobile display system 100. Having a more compact and more streamlined profile makes cordless and wireless mobile display system 200 more maneuverable compared to cordless and wireless mobile display system 100. Also, because cordless and wireless mobile display system 200 houses more of the signal wiring within its structural body, it can easily be transported from different locations within a single procedure room or to other surgical rooms entirely without the need to disconnect or reconnect any cables. Another consideration of note for a surgical environment is that having less surface area compared to cordless and wireless mobile display system 100, cordless and wireless mobile display system 200 presents less opportunities for its surfaces to become contaminated and having harmful germs then be transferred to an exposed patient during a surgical procedure.

Cordless and wireless mobile display system 200 includes display assembly 210 including a display panel 214, a mobile stand 202, a stem 204 that connects display assembly 210 with mobile stand 202, and a battery assembly 220. While cordless and wireless mobile display system 200 may include similar associated components 230 such as analyses and monitoring components, aspects of these components, entire portions of these components, or entire components may be incorporated into cordless and wireless mobile display system 200.

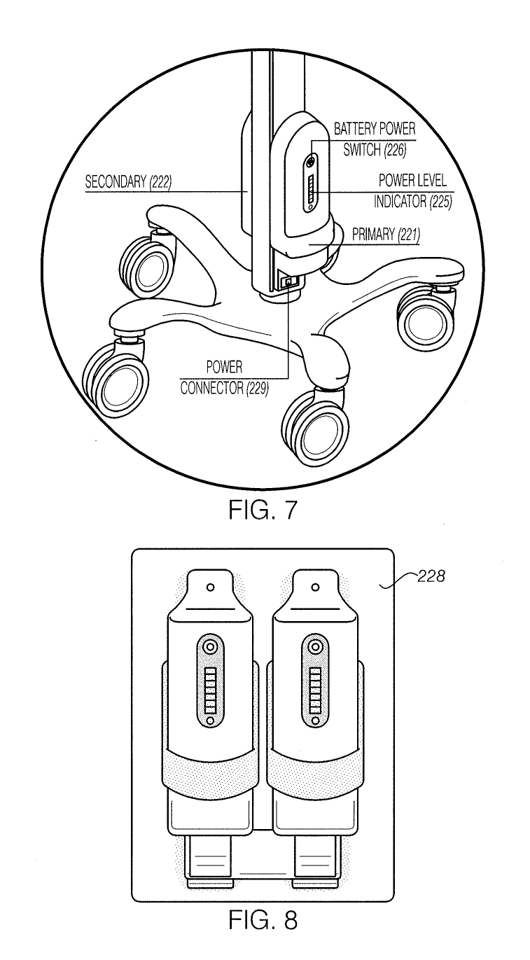

As previously mentioned, cordless and wireless mobile display system 200 includes both display assembly 210 and interface with battery assembly 220 that are now mainly retained within the body of cordless and wireless mobile display system 200. The majority of battery assembly 220 remains exposed and accessible to the user as shown in FIG. 7. Staying with FIG. 7, primary battery 221 and secondary battery 222 are positioned on stem 204. In this example, battery assembly 220 can be located on stem 204 in closer proximity to mobile stand 202 than to display 210, but in other examples, the battery assembly may be located anywhere along the stem of the cordless and wireless display system or even on the display or mobile stand. Next, cordless and wireless mobile display system 200 also includes a battery power switch 226. Similar to the previous embodiment described, battery power switch 226 is able to switch which power source (either primary battery 221 or secondary battery 222) supplies energy to the various components of cordless and wireless mobile display system 200. The ability to switch between the two batteries may be both manual and/or automatic. Thus, a user would be able to force either primary battery 221 or secondary battery 222 to supply the power to cordless and wireless mobile display system 200. But in standard operating mode, battery power switch 226 will switch between primary battery 221 and secondary battery 222 based on signals received from battery controller 223. Similar to the previous embodiment, battery controller 223 is in communication with sensors 224 that are able to determine if either power output for primary battery 221 or secondary battery 222 falls below a pre-determined output level. Once sensor 224 detects power output for the primary battery 221 below the pre-determined output value, battery controller 223 will signal to battery power switch 226 to open the power circuit loop that connects primary battery 221 and close the circuit loop to secondary battery 222 so that the system components are able to draw power from only the secondary battery 222. In some examples, the battery power switch also includes indicators as to which battery the system is drawing power from. Battery power switch may also include a central power switch for turning the entire system on or off.

Turning to FIG. 8, a charging unit 228 is shown. Charging unit 228 is able to plug directly into a power source such as a wall outlet. Charging unit 228 shown in FIG. 8 is able to charge two batteries at once or one battery at a time. In other examples, the charging unit may recharge more than two batteries at a time. The charging unit may also have indicators as to when each battery has been fully recharged.

Next, a power connector 229 may also be included with battery assembly 220. Power connector 229 may provide a backup if neither primary battery 221 nor secondary battery 222 are able to provide adequate power to the system. Power connector 229 may have standard coupling design that will interface with commercially available power cords and connectors.

Finally, another variation of the cordless and wireless display is a standalone wireless and cordless display having a primary and secondary battery assembly. In this variation, the display does not include a mobile stand. This cordless and wireless display may have couplers for hanging the display or stand for standing the display up at a desired location.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions.

For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A cordless wireless mobile display system for use in a surgical procedure room comprising:
a display;
a wireless receiver and a wireless transmitter in communication with the display;
a battery assembly that provides power to the display, the battery assembly comprising:
a primary battery, wherein the primary battery provides power to the display under normal operation; and
a secondary battery wherein the secondary battery is able to provide power to the cordless mobile display system upon the primary battery's output falls below a predetermined output value;
a battery controller;
a battery charging station; and
a mobile stand supporting the display, the receiver, the transmitter, the battery assembly, and the battery charging station.

2. The display system of claim 1, wherein the wireless receiver and the wireless transmitter is able to transmit and receive information in real time.

3. The display system of claim 1, wherein the battery controller is able to monitor the power output from the primary battery and the secondary battery.

4. The display system of claim 1, wherein the battery controller is able to switch power from the primary battery to the secondary battery when the primary battery's output falls below the predetermined output value.

5. The display system of claim 4, wherein the battery controller is able to switch power from the primary battery to the secondary battery within ten milliseconds once the primary battery power output falls below the predetermined output value.

6. The display system of claim 1, wherein the battery controller is able to charge the primary battery and the secondary battery.

7. The display system of claim 1, wherein the battery controller is able to measure and display remaining time left for either the primary battery or the secondary battery.

8. The display system of claim 1, wherein the system further comprising an external wireless receiver and an external wireless transmitter.

9. The display system of claim 1, wherein the system further comprises a battery power indicator configure to indicate when the power level of the primary and the secondary battery.

10. The display system of claim 1, wherein the system further comprises an alert indicator adapted to indicate when power is switched from the primary battery to the secondary battery.

11. The display system of claim 1, wherein the system further comprises at least one medically-used monitoring, sampling, or analyses component in wired or wireless communication with the display, wireless receiver and transmitter, and the battery assembly.

12. The display system of claim 1, wherein the mobile stand is supported by at least three wheels.

13. A cordless wireless mobile display system for use in an surgical procedure room comprising:
a display;
a wireless receiver and a wireless transmitter communicating with the display;
a battery assembly that provides power to the display, the battery assembly comprising:
a primary battery, wherein the primary battery provides power to the display under normal operation; and
a secondary battery wherein the secondary battery is able to provide power to the cordless mobile display system upon the primary battery's output falls below a predetermined output value;
at least one medically-used monitoring, sampling, or analyses component in wired or wireless communication with the display, wireless receiver and transmitter, and the battery assembly;
a battery controller, wherein the battery controller is able to:
monitor and detect the power output from the primary battery and the secondary battery;
switch power from the primary battery to the secondary battery within ten milliseconds when the primary battery's output falls below the predetermined output value;
charge the primary battery and the secondary battery; and
measure and display remaining time left for either the primary battery or the secondary battery
a battery charging station; and
a mobile stand having wheels for supporting wireless display, the components, the battery assembly, and the battery charging station.

14. The display system of claim 13, wherein the system further comprises a battery power indicator adapted to show the power level of the primary and the secondary battery.

15. The display system of claim 14, wherein the system further comprising an alert indicator configured to indicate when power is switched from the primary battery to the secondary battery.

16. The display system of claim 13, wherein the system further comprising a USB connection transferring information on the battery assembly status.

17. The display system of claim 13, wherein a communication received or transmitted by the display system occurs in real time.

18. The display system of claim 17, wherein the communication received or transmitted is a video image.

19. A cordless wireless mobile display system for use in a surgical procedure room comprising:
a display;
a wireless receiver and a wireless transmitter in communication with the display;
a battery assembly that provides power to the display, the battery assembly comprising:
a primary battery, wherein the primary battery provides power to the display under normal operation; and
a secondary battery wherein the secondary battery is able to provide power to the cordless mobile display system upon the primary battery's output falls below a predetermined output value;
a battery controller;
a battery charging station; and
a stand for keeping the display upright.

20. A cordless wireless mobile display system for use in an surgical procedure room comprising:
a display;
a wireless receiver and a wireless receiver communicating with the display;
a battery assembly that provides power to the display, the battery assembly comprising:
a primary battery, wherein the primary battery provides power to the display under normal operation; and
a secondary battery wherein the secondary battery is able to provide power to the cordless mobile display system upon the primary battery's output falls below a predetermined output value;
at least one medically-used monitoring, sampling, or analyses component in wired or wireless communication with the display, wireless receiver and transmitter, and the battery assembly;
a battery controller, wherein the battery controller is able to:
monitor and detect the power output from the primary battery and the secondary battery;
switch power from the primary battery to the secondary battery within ten milliseconds when the primary battery's output falls below the predetermined output value;
charge the primary battery and the secondary battery; and
measure and display remaining time left for either the primary battery or the secondary battery
a battery charging station; and
a stand for keeping the display upright.
